(19) United States
(12) Patent Application Publication
(10) Pub. No.: US 2007/0125392 A1
(43) Pub. Date: Jun. 7, 2007

(54) Systems and methods of accounting for surgical instruments and disposables

(75) Inventors: Stanley W. Olson JR., Germantown, TN (US); Jeffrey H. Nycz, Collierville, TN (US); Robert Varner, Germantown, TN (US); Steven M. Tethrake, Collierville, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SDGI Holdings, Inc.

(21) Appl. No.: 11/290,530

(22) Filed: Dec. 1, 2005

(51) Int. Cl. A61B 19/00

(52) U.S. Cl. 128/899

(57) ABSTRACT

A system and method for the preventing unintended surgical instruments and surgical disposables from remaining in the body cavity of a patient post surgically. Individual items are tagged with an RFID transponder tag preferably at the time of manufacture. Then, after an operation is complete, but prior to would closure, the body of the patient is scanned using an RFID reader device, such as, a hand held reader device to detect the presence of any latent RFID tags. As long as no tags are identified it is presumed that the body cavity is free of any unintended items. This avoids sponge counting and other overhead intensive methods of accounting for surgical items during a surgical procedure.

### FIELD OF THE INVENTION

Embodiments of the invention generally relate to radio frequency identification systems, and more particularly to systems and methods for accounting for surgical disposables and other surgical items and/or instruments during surgical procedures through RFID tagging and scanning of these items to insure that unintended items are not closed into the body cavity.

### DESCRIPTION OF RELATED ART

Modern surgical procedures typically utilize numerous specialized tools and equipment and involve teams of several personnel working simultaneously. Because of time limits of anesthesia, the patients health, the nature of the surgical procedure, changes in the patient's status, and lengthy shifts worked by medical personnel, the operating room environment is a dynamic and sometimes chaotic environment, hectic and reactionary. As a result, it is not uncommon for disposable items, such as gauze, sponges, and tamponades, and even re-usable items and surgical instruments, such as spreaders, retractors, needle holders, etc., to be inadvertently left in the body of a patient following surgery. The patient's retention of these items may increase the chances of infection or otherwise harm the patient's body. The likelihood of items being left in a patient's body is increased if the patient is overweight or if the procedure is an unscheduled emergency room procedure. Location and retrieval of items left within the patient's body cavity can unnecessarily prolong surgical procedures, increase the risk of infection, prolong anesthesia, contribute to suboptimal patient recovery and increase liability for hospitals, clinicians and even manufactures/suppliers. Frequently, sponges or other items not detected through a conventional type of accounting will remain undetected until days, weeks, and even years later and often not until the patient begins to suffer negative health effects.

One proposed solution to the problem of accounting for surgical disposables and other equipment and instruments is to perform pre and post operative item counts. For example, a person on the surgical team will be responsible for counting each surgical sponge prior to performing the procedure and then counting the used and unused sponges again afterwards to insure that each sponge is accounted for. This solution is suboptimal because it is prone to human error. Manual counting creates a heavy burden to accurately count and remember the number of items. Sometimes product packaging is defective and may include an inaccurate and misrepresentative disposable item count, that is the actual contents have too few or too many items, leading to a potential for unnecessary and lengthy searches and recounts or items being retained that are intended for removal. Also, this type of tracking of items is relatively time consuming. Suboptimal communications between members of the surgical team increases the likelihood of incorrect sponge counts. Productivity pressures in medical facilities such as hospitals may interfere with adoption of standards and may compromise implementation of counting standards. Furthermore, the perception that socialization, music, and conversation are acceptable because the patient is asleep can contribute to lapses in concentration and therefore inaccurate counts. Also, the stressful environment of the operating room with people frequently entering and leaving may require change in roles and responsibility and may tend to undermine accurate manual counts.

In light of the many shortcomings of manual item counts, other identification techniques have been proposed. Many surgical disposables and other items in use today are marked with radiopaque media (barium sulfate, radiopaque metals, etc.) so that they can be seen on imaging systems (x-ray, computerized tomography, magnetic resonance imaging, etc.). Especially when counts suggest that items are unaccounted for, perhaps retained within the wound and prior to closing a patient wound, the patient may be explored or imaged to search for retained disposables and other instruments, etc., that are not intended as therapeutic implants. In conjunction with or following negative exploration for retained items, the patient is imaged (x-rayed) on the operating table. While this solution does ameliorate some of the problems of manual counting, it still suffers from some significant drawbacks. One problem with X-ray detection is that the position of the patient is relatively limited during surgery—thus, optimal X-rays may not be obtained, thereby increasing the likelihood that items remain undetected. Also, the person performing the X-ray may not be familiar with what they are looking for and/or the imaging beam may be improperly positioned relative to the specific surgical field—thus, the human eye is still required to discover these items. Furthermore, items located deep within tissue or near prosthetic devices may be undetectable, especially if and when the prosthetic devices themselves are radiopaque and block the views of all items within the wound. Performing X-rays during surgery is obtrusive because it requires everyone to leave the operating room, and the inability to obtain immediate results from X-rays may prolong surgical procedures. Finally, because repeated and/or lengthy exposure to X-rays (radiation) is known to prolong wound healing and may even cause cancer, use of X-rays should be limited. Thus, radiopaque media are not an optimal means of accounting for surgical disposables and other surgical items.

Other methods of identifying surgical disposables beyond hand counting and X-ray tracking methods involve the use of 2D matrix symbols, bar codes, or other printed machine readable indicia. These systems require that each items scanned prior to use and then scanned again following use. The system reconciles the before and after counts to determine if any items are missing. These methods are undesirable because they still require, handling each item before use and saving each item after use. Also, after item become soiled with bodily fluids, such as blood, the indicia can become obscured making reads more difficult. Also, these systems are only able to determine that an item is missing, rather than specifically determining that an item is remaining in the body of a patient.

Radio frequency identification (RFID) systems use an RF field generator (reader) and a plurality of RFID transponder tags that store information about the goods and products to which they are attached. RFID tags are miniature electronic circuits that typically consist of a coil that acts as an antenna and a small silicon-based microprocessor with a memory, all encapsulated in a sealing material. RFID tags store identification information, usually in the form of an identification number that corresponds to an object or item to which the tag is attached. When a transponder tag enters an RF field generated by a reader device, the circuit of the tag becomes energized causing the processor to perform a data operation, usually by emitting a signal containing the processor's stored information. The basic structure and operation of RFID tags can be found in, for example, U.S. Pat. Nos. 4,075,632, 4,360,801, 4,390,880, 4,739,328 and 5,030,807, the disclosures of each of which are hereby incorporated by reference in their entirety.

RFID tags generally are formed on a substrate, such as, for example, paper, so that they can be easily applied to various items to be identified. The tags can include analog RF circuits, digital logic, and memory circuits. RFID tags also can include a number of discrete components, such as capacitors, transistors, and diodes. RFID tags are categorized as either active or passive. Active tags have their own discrete power source such as a battery. When an active tag enters an RF field it is turned on and then emits a signal containing its stored information. Passive tags do not contain a discrete power source. Rather, they become inductively or capacitively charged when they enter an RF field. Once the RF field has activated the passive circuit, the passive tag emits a signal containing its stored information. Passive RFID tags usually include an analog circuit that detects and decodes the interrogating RF signal, and that provides power from the RF field to a digital circuit in the tag. The digital circuit generally executes all of the data functions of the RFID tag, such as retrieving stored data from memory and causing the analog circuit to modulate to the RF signal to transmit the retrieved data. In addition to retrieving and transmitting data previously stored in the memory, both passive and active dynamic RFID tags can permit new or additional information to be stored in the RFID tag's memory, or can permit the RFID tag to manipulate data or perform some additional functions.

Thus, there is a need for an accurate and efficient automated system for accounting for surgical disposables and other surgical items that is not dependent upon manual counting, that does not suffer the shortcomings of x-ray techniques or suffer the shortcomings of bar coding or other machine readable indicia-based tracking systems.

The description herein of various advantages and disadvantages associated with known apparatus, methods, and materials is not intended to limit the scope of the invention to their exclusion. Indeed, various embodiments of the invention may include one or more of the known apparatus, methods, and materials without suffering from their disadvantages.

### SUMMARY OF THE INVENTION

Based on the foregoing, it would be desirable to provide a system and method for accounting for surgical disposables and other surgical items that is not prone to the errors associated with manual counting. It also would be desirable to provide a system and method for accounting for surgical disposables that is able to penetrate tissue harmlessly, but that also is less dependent upon human detection. In various embodiments, such a system and method is based on providing surgical disposables and other surgical items with RFID tags and using an RFID scanner to detect the presence of these items within the body cavity prior to closure.

RFID technology is particularly well suited to the problem of accounting for surgical disposables and other surgical items. One benefit is that RF waves can easily penetrate human tissue, thus allowing medical personnel to “see” into the body cavity. Another benefit is that these waves are at power spectral density levels that are relatively harmless compared to X-rays. Still another benefit is that detection is automated rather than dependent on human interpretation of X-ray film. A reader device can read several tags at once, count them, and even obtain stored in formation in the tag, such as, for example, identification numbers, names, etc. Yet another benefit is that reading devices can be hand held allowing for relative easy scanning of the patient's body or they can be placed at appropriate positions to permit continuous scanning during the procedure in the operating room.

It is a feature of various embodiments of the invention to provide a wireless system for accounting for surgical disposables and other items based on RF identification techniques. Another feature of various embodiments of the invention provides a method for discovering latent surgical disposables and other surgical items remaining in the body cavity of a patient. Yet another feature of various embodiments of the invention is to provide a method for accounting for surgical disposables and other surgical items that is based on RFID technology and that can alert medical personnel when an accounting error occurs.

To achieve the above-noted features, and in accordance with the purposes as embodied and broadly described herein, one exemplary embodiment provides a method of preventing surgical items from remaining in a patient's body post operatively. The method according to this embodiment comprises attaching an RF transponder tag to each of a plurality of surgical items prior to use in a surgical procedure, and scanning the patient's body at least once prior to closure with an RFID reader device.

In accordance with another exemplary embodiment, a method of accounting for surgical items during a surgical procedure is provided. The method according to this embodiment comprises scanning a group of RFID-tagged items with an RF reader device prior to performing a surgical procedure on a patient, performing the surgical procedure utilizing at least one of the RFID-tagged items, scanning used and unused items after performing the surgical procedure, and scanning the patient's body with the RF reader device before closing to determine if any items are unaccounted for.

In accordance with a further exemplary embodiment, a system for preventing surgical items from remaining in the body of a patient post-operatively is provided. The system according to this embodiment comprises a plurality of surgical items, each item comprising an RFID tag attached thereto, and an RFID reading device adapted to identify the presence of each of the plurality of surgical items by reading data from the RFID tags.

These and other embodiments and advantages of the present invention will become apparent from the following detailed description, taken in conjunction with the accompanying drawings, illustrating by way of example the principles of the invention.

### BRIEF DESCRIPTION OF THE DRAWINGS

Purposes and advantages of the embodiments will be apparent to those of ordinary skill in the art from the following detailed description in conjunction with the appended drawings in which like reference characters are used to indicate like elements, and in which:

FIG. 1 is a perspective view of several different surgical disposable items according to various embodiments;

FIG. 2 is a cut away perspective view of an RFID tagged surgical disposable item according to various embodiments;

FIG. 3 is a perspective view of a surgical procedure site on a patient's body according to various embodiments;

FIG. 4 is a flow chart outlining the steps of a method for preventing surgical items from remaining in a patient following surgery according to various embodiments.

FIG. 5 is a flow chart outlining the steps of a method for preventing surgical items from remaining in a patient following surgery according to various embodiments.

### DETAILED DESCRIPTION

The following description is intended to convey a thorough understanding of the embodiments described by providing a number of specific embodiments and details involving systems and methods for accounting for surgical disposables and other items and surgical instruments using RFID technology. It is understood, however, that the present invention is not limited to these specific embodiments and details, which are exemplary only. It is further understood that one possessing ordinary skill in the art, in light of known systems and methods, would appreciate the use of the invention for its intended purposes and benefits in any number of alternative embodiments, depending upon specific design and other needs.

As used herein, the expressions “RFID tag” and “RFID transponder tag” will refer to any active or passive type of electronic data storage device, read-only or read and write, that is wirelessly activated in the presence of a radio frequency (RF) field, including any currently available inductively coupled RFID tags, capacitively coupled RFID tags and even future RF-type tags not yet available. This includes tags operating in the 125 kHz, 13.56 MHz, 868-927 MHz, 2.45 GHz and 5.8 GHz frequency bands as well as other suitable frequency bands. Also, the tag may be a silicon-type IC tag, a printed tag printed with a conductive ink-based printing process or a tag formed by other suitable means. It should be appreciated that in various embodiments, because the tag will be attached to items in contact with living tissue, that the tag should be encapsulated using a suitable biomedical encapsulation process utilizing stable, inert materials known not to cause adverse reactions to surrounding tissue. Such materials are well known in the surgical arts. Therefore a detailed discussion of them has been intentionally omitted.

As used herein, the expression “surgical disposables,” will refer to any type of surgical or medical disposable such as, for example, sponges, tamponades, gauze, etc., commonly used in surgical procedures. Also, the expression “surgical item” as used herein may be substituted for “surgical disposables” or may also refer to other non-disposable surgical equipment, such as, for example, surgical instruments, clamps, retractors, forceps, spreaders, scissors, scalpels, etc. Though the specification is written primarily in the context of surgical disposables, it should be appreciated that the principles discussed herein may also be used with any other surgical items themselves, or together with the surgical disposables that may be inadvertently left within the body cavity of a patient following surgery.

As used herein, the term “patient” will refer to a person or other creature undergoing a surgical procedure. Though the specification is written primarily with reference to human patients, it should be appreciated that the principles of the invention may be applied equally to veterinarian surgery on animals.

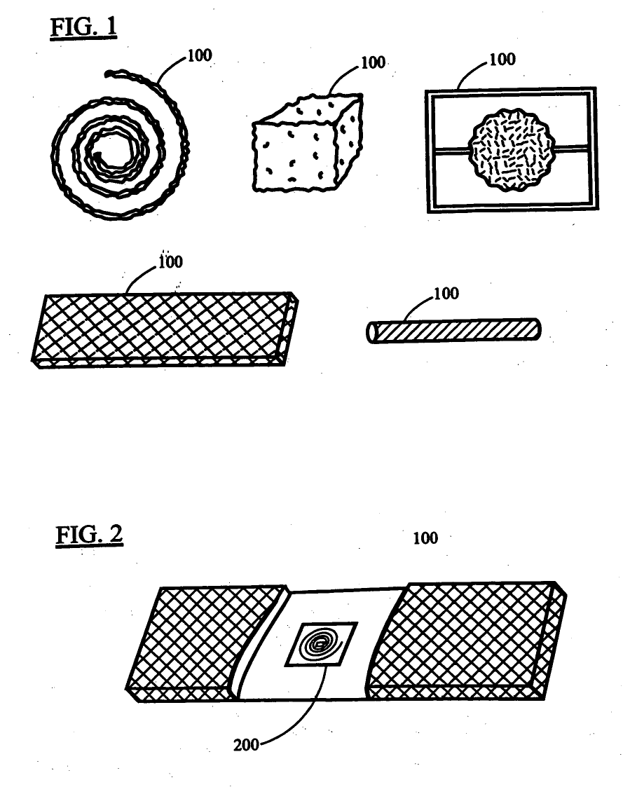

Referring now to FIG. 1, several different surgical disposables 100 are illustrated, including sponges, gauze and tamponades. As discussed herein, these items 100 are typically used to absorb body fluids, such as blood, so that they don't obscure the surgeon's view of the relevant body area during surgical procedures. The danger of having them remain in the patient's body stems from the fact that they are inserted into the site of the opening and may become concealed by tissue and/or organs, especially when saturated with blood or other body fluid.

In various embodiments, prior to surgery, an inventory of surgical disposables 100 will be created to accommodate the anticipated demand during surgery, depending upon the nature of the procedure. In various other embodiments, these items 100 will be taken as needed from a bulk container. As items are consumed or the useful life is reached (sponges are saturated with blood and no longer keep the field dry or contribute to hemostasis), they are withdrawn and accoutned for as they exit the body and the surgical field, being subtracted from the preoperative inventory of disposables and other surgical items.

In various embodiments of this invention, each surgical disposable 100 will contain an integral or attached RFID tag. In order to make the tags as inexpensive as possible, it may be desirable that the tags are passive type tags. However, other tag types can be used within the context of this invention. The RFID tag may be attached to the item 100 using an adhesive material or embedded within the item 100. In a preferred embodiment, in order to maintain sterilization of the items 100, the tag and item 100 will be joined during a stage of the item's manufacture. However, it should be appreciated that that manual application (e.g., by tying, stapling, taping, etc.) of the tag to the item 100 also is within the scope of various embodiments of this invention.

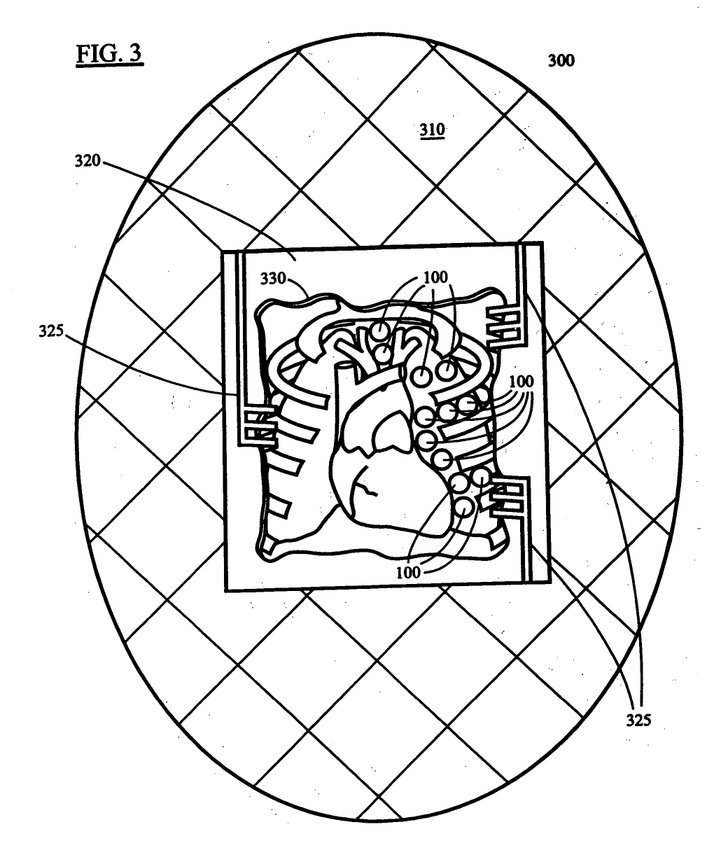

Referring now to FIG. 2, a cut away view of a surgical sponge 100 is illustrated. In FIG. 2, the cut away portion reveals an RFID transponder tag 200. As discussed above, any suitable RFID tag 200 may be utilized with this invention. Because RFID tags in general are well known in the art, a detailed discussion of the tag 200 itself has been intentionally omitted. Typically, RFID tags comprise a processor, a memory and a coil antenna. In various embodiments, because the tag 200 is located in an object used to absorb bodily fluids, the tag 200 will preferably be coated with a water impermeable film or other sealant to preserve the integrity of the tag's electronic components. However, at least with respect to surgical disposables, it is not necessary that the tag be ruggedized to withstand the rigors of sterilization or other high heat environments, because these items are disposed of after a single use. However, as noted above, the tag should be encapsulated in a suitable biomedical material that will ensure that the tag materials to do cause any adverse reactions to living tissue that it may come into contact with.

In the embodiment illustrated in FIG. 2, the tag 200 is shown as attached to a layer of the surgical disposable, in this case, a flat sponge. However, other surgical disposables may lend themselves to different means of attachment. For example, a non-flat object, such as a tamponade or cotton ball may require that the tag be embedded rather than surface mounted. The specific location of tag with respect to the item to be identified is not critical to the embodiments of the invention. Rather, various designs and techniques may be used for attaching the RFID transponder tag to the various surgical disposables or other surgical items.

In certain embodiments, the RFID transponder tag 200 is preprogrammed with identification information for the item that it will be attached to. Therefore, once tagged, the item may be wirelessly inventoried by activating the RFID transponder tag 200 using a suitable RF reader device. However, in various other embodiments, the tag will not be preprogrammed but rather is programmed after being attached to the object using an RFID writing or reading/writing device. Because RFID reader devices and reader/writer devices are well known in the art, a detailed discussion of such devices has been intentionally omitted. The transponder tag 200 according to the preferred embodiment is compatible with any suitable reader and/or reader/writer devices whether hand held, stationary, fixed or otherwise configured.

In various embodiments, the tag processor 130 contains read only memory. However, in various other embodiments, the tag processor 130 contains a read and write memory. In still further embodiments, the tag processor 130 contains a portion of memory that is permanent and portion that may be written to.

Referring now to FIG. 3, a perspective view of a surgical procedure is illustrated. The perspective view 300 shows a portion of a patient's body 320 exposed through a sheet 310 to highlight the location of surgery. An opening 330 is made in the body cavity of the patient and is maintained using spreaders 325, which maintain the size of the opening 330. In the example shown in FIG. 3, a chest cavity of a patient is exposed through the surgical opening 330, such as, for example, during a bypass surgery procedure. During the surgical procedure, in addition to the spreaders 325 inserted into the patient's body cavity, several surgical disposables 100 are present in the opening 330 to absorb fluids and permit the medical team to see the relevant tissue/organ structure. In a typical procedure, these items remain in the opening 330 until the procedure is finished, or are swapped out with replacement items as necessary. Thus, using conventional item counting practices, any items removed must be individually counted. If the item is replaced there is no net effect on total item count, however, this may lead surgical personnel to believe that there are unaccounted for items or alternatively that there are not when actually there are. Furthermore, items removed from the body cavity must be kept in such a way that they can be discerned from one another for counting purposes.

Using the techniques of the present invention, if a count is being maintained, the items can simply be dumped into a container together because RFID reading devices enable bulk reading of a several items at once. Alternatively, if no count is being maintained, the items may be discarded as they are removed from the body. A check for latent items will be performed by simply holding an RFID reading device over the surgical opening 330 or subjecting the opening to an RFID field using techniques known in the art. If no RFID transponder tags are detected, then surgical personnel can have a high confidence that there are no latent items present. Otherwise, the reader will identify the type of item remaining and the surgical personnel will use this information to locate the item. If difficulty in locating the item is experienced, then either X-ray or metal detecting techniques may be used to specifically target the missing item. Because the RFID tag will typically contain metal components, separate radiopaque coatings usually are not necessary.

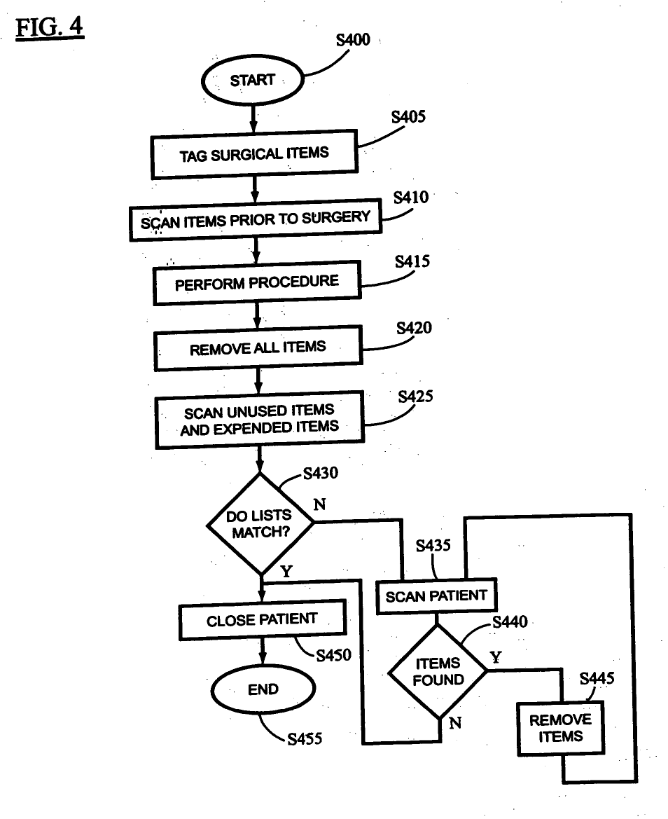

Referring now to FIG. 4, a flow chart illustrating a method for preventing surgical disposables and other items from remaining in the body cavity of a patient postoperatively in accordance with various embodiments is illustrated. The method begins in S400 and proceeds to S405 where each surgical disposable or surgical item is tagged with an RFID transponder tag. As discussed herein, the tagging step may be a manual step performed by medical personnel prior to surgery or may be performed during the manufacture of the item, requiring no additional work by personnel in the operating room.

After tagging in S405, operation of the method proceeds to S410 where the items to be used in surgery are scanned using an RFID reading device. In various embodiments, a count list will be established prior to surgery by scanning the items available for use in surgery. This count list will be stored in a memory of the reading device and then matched against another list created by scanning items expended during surgery and any unused items. Next, operation goes to S415 where the surgical procedure is performed utilizing some of the items scanned in S410. After the procedure, operations proceed to S420 where all items placed in the patient's body are removed and retained. Then, in S425, the items removed in S420 as well as any unused items are scanned. The list created by the scanning of S425 is compared against the list created in S410. Based on this comparison, in S430, a determination is made whether the lists match. If so, processing goes to S450 where the patient is surgically closed. Otherwise, processing goes to S435 where the patient is scanned using the RFID reader to determine if the missing item(s) is still within his/her body cavity. Next, in S440, if it is determined that the missing item(s) is not in the patient's body, that is no RFID transponder tags are found when the patient's body is scanned, it is presumed that no items remain in the patient's body. Alternatively, if any item(s) is found, operation proceeds to S445, where the item is removed from the patient's body. Next, operation repeats S435 where the patient's body is once again scanned with the RFID reader to insure that all items have been removed.

It should be appreciated that although not shown in FIG. 4, if, at S430 it is determined that one or more items are missing, and the subsequent scanning of the patient does not reveal any items, a secondary identification process may be used such as, for example, an X-ray or metal detecting process to insure that no items remain.

After it has been determined that all items are accounted for, operation proceeds to S450 where the patient is closed. Operation of the method terminates in S455. Because before and after lists are determined using the method of FIG. 4, the method may also communicate with the medical facility's inventory tracking system to deduct the items from inventory and update for ordering purposes.

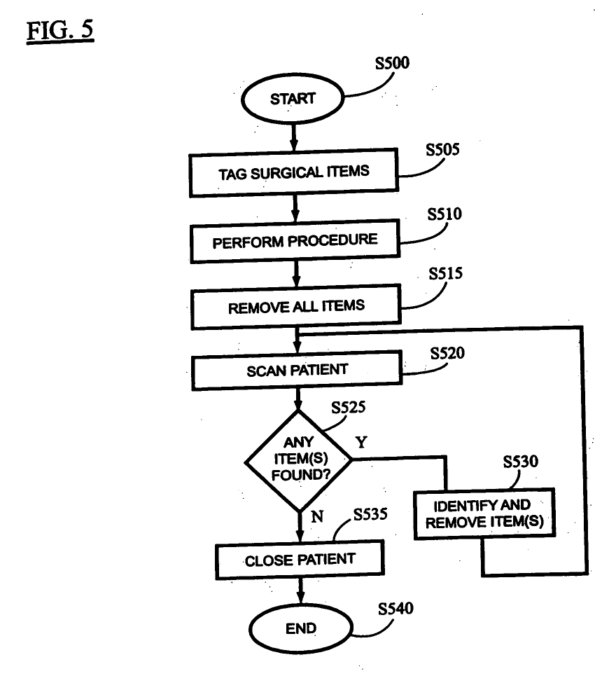

Referring now to FIG. 5, a flow chart illustrating another method or preventing surgical disposables and other items from post operatively remaining in the body of a patient is illustrated. The method begins in S500 and proceeds to S505 where each of the individual surgical items, disposables and/or other items, are tagged with an RFID transponder tag. As discussed above in the context of the method outlined in FIG. 4, this may be performed prior to surgery by medical personnel or performed during the manufacturing process of the items.

Operation of the method proceeds to S510 where the surgical procedure is performed using at least one of the items tagged in S505. Then, in S515, after the procedure is finished, all items inserted into the surgical opening are removed and discarded. Then, in S520, the patient is scanned using an RFID reader device. Next, in S525, a determination is made as to whether any items have been identified in the patient's body. If not, operation of the method proceeds to S535. Otherwise, operation goes to S530, where any remaining items are identified and removed. After S530, operation of the method returns to S520 where the patient is once again scanned to insure all items have been removed.

After it has been determined that all items have been removed, operation of the method goes to S535 where the patient is surgically closed. Operation of the method ends in S540.

Through the use of the methods outlined in FIGS. 4 and 5, medical personnel may be able to insure with a high level of confidence that surgical disposables and/or other items do not remain in a patient postoperatively. This will reduce risk liability for medical practitioners and enhance chances for normal patient recovery. Because hand held (and optically wireless) RFID reader, or RFID readers positioned at or near the operating table preferably are used in the embodiments, there is no need to physically remove RFID tagged items from the immediate operating area and place them in a relatively remote RFID reader.

It should be appreciated that the various systems and methods for preventing surgical disposables an other surgical instruments from remaining in the body of a patient post operatively may have to be performed post operatively so as to prevent interference between the RFID reader and other sensitive medical equipment. Also, the system may be forced to operate in a more limited frequency band as opposed to open frequency bands in order to prevent interference.

While the foregoing description includes many details and specificities, it is to be understood that these have been included for purposes of explanation only, and are not to be interpreted as limitations of the present invention. Many modifications to the embodiments described above can be made without departing from the spirit and scope of the invention.

What is claimed:

1. A method of preventing surgical items from remaining in a patient's body post operatively comprising:
attaching an RF transponder tag to each of a plurality of surgical items prior to use in a surgical procedure; and
scanning the patient's body prior to closure with an RFID reader device.

2. The method according to claim 1, wherein attaching is performed during manufacture of each of the plurality of surgical items.

3. The method according to claim 1, wherein surgical items comprises items selected from the group consisting of surgical disposables and surgical instruments.

4. The method according to claim 1, further comprising generating an alert if scanning the patient's body discovers the presence any RFID tags.

5. The method according to claim 1, wherein scanning the patient's body comprises passing a handheld scanner over the surgical site on the patient's body.

6. The method according to claim 5, further comprising identifying and removing any detected items prior to closure of the patient.

7. The method according to claim 5, further comprising, generating a data record based on the results of the scanning and storing the data record on a data processing system.

8. The method according to claim 7, wherein storing the data record comprises storing the data record as part of the patient's medical record.

9. A method of accounting for surgical items during a surgical procedure comprising:
scanning a group of RFID-tagged items with an RF reader device prior to performing a surgical procedure on a patient;
performing the surgical procedure utilizing at least one of the RFID-tagged items;
scanning used and unused items after performing the surgical procedure; and
scanning the patient's body with the RF reader device before closing if any items are unaccounted for.

10. The method according to claim 9, wherein scanning a group of RFID-tagged items comprises scanning with an RF reader device and storing an electronic list of the items based on an item identification number read by the reader device.

11. The method according to claim 10, wherein scanning used and unused items comprises scanning the items with an RF reader device and comparing the identification numbers of the scanned items against those in the electronic list.

12. The method according to claim 9, further comprising generating an alert if any items are unaccounted for.

13. The method according to claim 12, wherein the step of generating an alert comprises generating an alert selected from the group consisting of an audible alert, a visual alert, and combinations thereof.

14. The method according to claim 7, further comprising generating a data record from the results of at least one of scanning the group of RFID-tagged items, scanning used and unused items after performing the surgical procedure, and scanning the patient's body, and storing the data record on a data processing system.

15. The method according to claim 14, wherein storing the data record comprises storing the record in a medical supply inventory system of a medical facility.

16. The method according to claim 14, wherein storing the data record comprises storing the record in as part of the patient's medical record.

17. A system for preventing surgical items from remaining in the body of a patient post-operatively comprising:
a plurality of surgical items, each item comprising an RFID tag attached thereto; and
an RFID reading device adapted to identify the presence of each of the plurality of surgical items by reading data from the RFID tags.

18. The system according to claim 17, wherein each surgical item is a member of a set of surgical items prepared for use in a surgical procedure.

19. The system according to claim 17, wherein the RFID reading device is adapted to identify to a user any RFID tags located in a patient's body.

20. The system according to claim 17, wherein the RFID reading device is adapted to compare a list of items identified preoperatively to a list of items postoperatively and to generate an alert if the two lists do not match.

21. The system according to claim 20, wherein the alert is an alert selected from the group consisting of a visual alert, an audio alert, and combinations thereof.

22. The system according to claim 17, further comprising a data storage device adapted to store as a data record data captured by the RFID reading device.

23. The system according to claim 22, wherein the data storage device is adapted to store data captured by the RFID reading device as a data record in a medical facility inventory database.

24. The system according to claim 22, wherein the data storage device is adapted to store data captured by the RFID reading device as part of a patient's medical record.
